(19) United States
(12) Patent Application Publication
(10) Pub. No.: US 2026/0257003 A1
(43) Pub. Date: Sep. 3, 2026

(54) SUCTION STENT

(71) Applicant: VAC Stent GmbH, Fulda (DE)

(72) Inventors: Constantin Heiss, Tübingen (DE); Timo Wächtersbach, Hünfeld (DE)

(73) Assignee: VAC Stent GmbH, Fulda (DE)

(21) Appl. No.: 18/995,653

(22) PCT Filed: Feb. 8, 2024
(86) PCT No.: PCT/EP2024/053256

§ 371 Date: Jan. 16, 2025

(30) Foreign Application Priority Data
Feb. 16, 2023 (WO) PCT/EP2023/053962

(51) Int. Cl. A61M 1/00

(52) U.S. Cl. CPC A61M 1/915; A61M 1/916; A61M 1/92; A61M 2207/00; A61M 2210/106; A61M 2210/1064

(57) ABSTRACT

A stent for introduction into a hollow organ of a human or animal body, in particular the gastrointestinal tract includes a stent body being resilient in a radial direction and having a wall defining an inner fluid passageway from one end of the stent body to a longitudinally opposing end of the stent body, and a resilient porous layer. The porous layer covers an outer surface of said wall along its entire circumference and along a predefined section in a longitudinal direction of said stent body. The porous layer and the wall are adapted for applying a negative pressure toward a portion of the hollow organ. The wall forms a liquid-tight barrier separating the inner fluid passageway and the porous layer, at least longitudinally opposing end faces of the porous layer are covered by a liquid-tight cover being connected to the respective longitudinal end of the wall.

### CROSS REFERENCE TO RELATED APPLICATIONS

This application is the U.S. National Stage of PCT/EP2024/053256 filed Feb. 8, 2024, which claims priority to PCT/EP2023/053962 filed Feb. 16, 2023, the entire contents of which are incorporated herein by reference in their entirety.

### TECHNICAL FIELD

The present invention relates to a stent for introduction into a hollow organ, in particular the gastrointestinal tract, of a human or animal patient. The stent may particularly be suitable to provide a vacuum sealing of leaks to a particular anatomic region in the hollow organ, e.g. for the treatment of local anastomosis insufficiencies.

### BACKGROUND

Leaks in surgical sutures (anastomoses) in the gastrointestinal tract form a serious health risk and hence constitute one of the most significant complications after surgery in the abdominal region. In the event of leaks, the contents of the stomach or intestine pass into the abdominal cavity and thus lead to peritonitis, a fatal event in about 20% of the incidents. Treating a leak is dependent on its location and the character of the pathophysiology upon leakage of the escaped contents of the intestine. Healing of the suture may be delayed and the functional result of the surgery, e.g. continence, may be impaired. Frequently, considerably more invasive measures such as a surgical intervention with removal of the intestinal continuity and fitting of a colostomy are required for saving the patient's life. The process of fitting a colostomy is reversed for only a fraction of affected patients.

Attempts to seal anastomosis insufficiencies, e.g. by using an endoscopically placed endoluminal covered stent or other conventional stents, was found to be frequently unsuccessful for adequately sealing the suture in hollow organs. That finding may generally be attributed to the incongruency of the applied stent with the irregularly shaped intestinal wall. Self-expandable stents with high restoring forces cannot be used for complete sealing in the region of leaky sutures either. They may rather elicit further damage at the site of the suture or may even trigger a bursting of the suture.

Even if, in exceptional cases, complete sealing of the defect is finally achieved, it is known that the contents, e.g. from the hollow organ that have entered the region of the suture, e.g. the contents of the intestine, cannot be drained away. Formation of an abscess at the suture site almost inevitably occurs, in particular in the gastrointestinal tract, thereby leading to further aggravation of the local pathological condition and the medical status of the patient.

To improve the sealing effect exerted by a stent towards the wall of the hollow organ, e.g. the intestine, the implementation of a porous foam material was suggested, which may be arranged at an exterior of a stent body and may be held in place by means of radially outward pressure exerted by the stent body. Moreover, drainage of detrimental contents from the respective hollow organ may be applied. A cannula at an exterior side of the stent body, e.g. within the porous material may be foreseen. By applying a lower than atmospheric pressure, an improved sealing of the suture may be envisaged. That approach may even allow the surgeon not to necessarily suture the lesion.

The usage of stents, however, was found to typically imply the occurrence of local tissue in-growth. In particular, the porous material and the typically mesh-like stent body appear to be susceptible to tissue in-growth. The use of a mesh-like stent body may furthermore requires significant extra efforts for the manufacturing of the stent. In particular, the arrangement and the fastening of the stent components to each other may require intricate manufacturing efforts to ensure that their relative position to each other is maintained upon deployment and/or application of a drainage function. Furthermore, it may be challenging in terms of the stent's design and its manufacturing to ensure that the mesh-like stent body is liquid-tight.

Therefore, a need exists to facilitate stent manufacturing and to further improve the characteristics of stents in terms of patient safety and treatment efficacy.

### SUMMARY

Starting from the known prior art, it is therefore an object of the present invention to provide a stent which effectively seals local defects, e.g. leaky surgical sutures. Advantageously, the stent structure provides a reliable and intimate connection with the hollow organ body site to be sealed and/or to be treated so as to effectively avoid leakage towards or from the anatomy to be treated. Preferably, any such stent also enables effective removal of any body fluids accumulated at the defective site, in hollow organs of the human or animal body. Furthermore, such a stent is preferably manufactured by a less costly and elaborate production process.

This object is achieved by the stent of the present invention as disclosed herein. Preferred embodiments are depicted by the description and the Figures.

Accordingly, a stent for introduction into a hollow organ of the human or animal body, preferably into the gastrointestinal tract, in particular the intestine, is disclosed. The inventive stent comprises a stent body being preferably resilient in a radial direction and having a wall defining an inner fluid passageway from one end of the stent body to a longitudinally opposing end of the stent body. It further comprises a resilient porous layer. Both the porous layer and the wall are adapted for the applying a negative pressure at the hollow organ implantation site. The porous layer covers an outer surface of said wall along its entire circumference and along a predefined section in a longitudinal direction of said stent body. According to the invention, the wall or the stent body forms a liquid-tight barrier separating the inner fluid passageway and the porous layer. According to the invention, the stent wall itself thus exhibits liquid-tight property. It is not required to cover the stent body e.g. a mesh-like stent body by a liquid-tight layer. At least the longitudinally opposing end faces of the porous layer are covered by a liquid-tight cover material being connected to the respective longitudinal end of the wall.

By providing the continuous wall structure in a liquid-tight manner, a sealing function with respect to the fluid passageway and towards the exterior is ensured, in particular when being deployed. The inventive stent renders the application of additional coatings, embedded structures, or foil material to provide a sealing obsolete. Such additional components may be effectively omitted as a result of the sealing function provided by the wall (or the stent body, respectively) of the inventive stent. Thereby, manufacturing of the inventive stent may be significantly facilitated. Moreover, potential sealing discontinuities may be effectively avoided.

The liquid-tight wall structure also impairs undesired in-growth of surrounding local tissue into the fluid passageway. The liquid-tight configuration of the wall has the further advantage that body fluids passing through the stent passageway are not conveyed or diffused towards the surrounding tissue at the implantation site, e.g. may not invade surrounding organs or enter into the blood circulation, e.g. at the suture or at the site of leakage. In particular, the presence of anastomoses in the surrounding tissue does not imply or at least reduces the risk of sepsis, by using the inventive stent. Provision of the liquid-tight wall structure allows for removal of the stent without generating tissue damage or at least without aggravating such tissue damage. Finally, the inventive stent facilitates wound healing in the surrounding tissue.

The wall structure of the body improves mechanical stability of the stent. In particular, its wall structure may be configured such that its resilience is larger in the radial direction compared with the resilience in the longitudinal direction. The stent's resilience may also be essentially limited to the radial direction. The stent body is preferably of essentially cylindrical or tubular shape. It may, however, also comprise other cross-sectional shapes, such as an ellipsoid shape, at least for one or more sections of the stent body. The cylindrical or tubular shape may further improve the mechanical stability of the body and for the stent as a whole. The body is preferably an elongated body with an essentially continuous longitudinal extension. Still, it may exhibit one or more curvatures, which may e.g. establish a geometry of the stent corresponding to a particular hollow organ structure, e.g. the sigmoid, which is to be supported or sealed.

Radial resilience of the wall facilitates compression of the stent in order to collapse the stent for deployment at the target tissue, i.e. for delivery to a target lesion or suture via a suitable delivery system and catheter. In other words, contrary to known incompressible stent bodies the radial resilience may enable a significant compression so as to reduce the dimensions or diameter of the stent to facilitate insertion into the hollow organ and deployment at the target tissue. Providing resilience or a level of radial flexibility furthermore supports the stent's adaptability to the respective anatomic structure at the site of its application, e.g. to the intestinal wall or to the intestinal wall at a specific region of the intestine. Depending on the dimension of the stent body, the resilience of the stent is thereby held in place e.g. by means of radially exerting forces. The stent body may also be configured to be self-expanding, e.g. it may be formed of a self-expandable plastics material.

The luminal diameter of the stent according to the invention, i.e. an inner diameter of the wall of the stent body, is preferably in the range of about 10 to 50 mm, preferably 15 to 35 mm, in particular 15 to 30 mm, most particularly preferably it is about 20 to 30 mm, such as 23 to 26 mm (for example in applications in the colon region) or about 10 to 20 mm, such as 10 to 15 mm (for example for use in the esophagus). In any case, the diameter of the stent is chosen such that, depending on the area of application, passage of corresponding material through the respective hollow organ—e.g. passage of chyme or stool in the case of the intestinal tract—is not obstructed.

The fluid passageway of the stent body or, at the site of its deployment, the stent body (referred to analogously throughout the present invention) may be understood as a through-channel or inner cavity with opposing openings. The stent body hence forms a hollow body having a lumen. It is open-ended in the longitudinal direction, such that a (e.g. viscous or semi-viscous) fluid may enter the passageway via one end and may exit the passageway via the opposing end.

Preferably, the wall of the stent body is fluid-tight and/or air-tight, preferably fluid-and air-tight. The stent may be equipped with drainage means, such as a cannula. Drainage means may be arranged outside of the wall. A negative pressure, i.e. a sub-atmospheric pressure or a vacuum, may be applied in the space between the wall of the stent and the inner wall of the hollow organ. Any contents leaking into this intermediate space (between the outer stent wall and the inner wall of the hollow organ) may hence be effectively drained by means of a negative or suction pressure. A (moderate) vacuum may furthermore facilitate sealing of a lesion or suture, thereby expediting the healing process. In particular, the wall of the stent may be configured to withstand negative pressures from −60 mmHg to −200 mmHg, preferably from −80 mmHg to −125 mmHg. The stent according to the invention is hence preferably configured as a suction stent.

The resilient porous layer is preferably shapeable and/or compressible. In the absence of compressive forces, the porous layer typically returns to its original non-compressed state. The porous layer may enclose the stent body and may e.g. be tubular-shaped having a central through-hole accommodating the stent body. The porous layer may be formed of a closed-pore material, typically in the form of a foam, or an open-pore material, i.e. in the manner of a sponge. Preferred porous layer materials are plastics material foams, for example including or consisting of silicone, polyurethanes, polyvinyl alcohols or mixtures of such plastics materials.

The porous layer preferably comprises a thickness of about 5 mm to about 20 mm, preferably of about 5 mm to about 12 mm or about 5 mm to about 10 mm. Its dimensions are dependent on the anatomic dimensions encountered at the application site and the structural constraints, e.g. the required resilience or the structural constraints for filling the space or volume between the stent body and the inner wall of the hollow organ.

The stent body and/or the stent according to the invention as a whole is preferably completely resilient, compressible, and, optionally, expandable. In particular, the stent body or the stent as a whole is not incompressible such that it may be compressed when e.g. being introduced into and delivered by a catheter. Complete resilience may allow the stent to be positioned at the application site. The inventive stent, in particular a stent based on complete resilience may be suitable for being positioned in the gastrointestinal tract, preferably the esophagus, intestine, primarily rectum, sigma, colon descendens or colon transversum, by conventional application measures.

The extension of the porous layer preferably corresponds to the longitudinal extension of the body. Such an embodiment renders the entire outer wall to be covered by the porous layer. Thereby, a sealing function may be optimized with regard to the surface area of the target tissue to be treated. A fitting, securing, and/or proper positioning of the stent may be improved. However, the wall of the stent body may comprise extensions at one or both of its longitudinal end portions for other embodiments. As a result, the wall of the stent body may only be partially covered with the porous layer.

The wall of the stent body and/or the porous layer is/are preferably formed of a plastic material or rubber material. Preferably, both the wall of the stent body and the porous layer are formed of a plastic material or rubber material. The provision of a plastic or rubber material may facilitate the resilience of the stent as a whole. These materials may also ensure that the wall is formed as a liquid-tight barrier. The plastic material or rubber material may also contribute to an improved adaptability or conformability of the stent body wall with regard to the inner wall of the hollow organ, establishing improved sealing and securing of the position of the stent.

The materials of the stent body and the porous layer are preferably (fully) biocompatible. Thereby, the deployment and presence of the stent at the target tissue, i.e. the hollow organ, does not trigger any or at least no significant adverse foreign-body reactions.

The porous layer may be formed of or may comprise one or more plastics material foams, for example including or consisting of silicone, polyurethanes, polyvinyl alcohols or mixtures of such plastics materials. The wall of the stent body may be formed of or may comprise a plastics or polymer material, e.g. being selected from the group comprising polyurethanes and latex.

Preferably, the wall of the stent body and/or the porous layer are formed of a silicon-based material, preferably comprising or consisting of silicone. Silicon-based materials are particularly advantageous in terms of their ease of handling for manufacturing. For example, materials such as PDMS or silicone, may be easily formed using a mold, allowing them to cure for a predefined curation time. Silicon-based materials furthermore exhibit excellent biocompatibility. Among the larger number of silicon-based materials, a material may be chosen, which exhibits the required level of resilience.

In an embodiment of the invention, the wall of the stent may be silicon-based and the porous layer may be formed of another different material, e.g. be based on one or more polyurethane foam materials. In other words, the wall of the stent body and the porous layer may be formed of distinct materials or material compositions.

In a preferred embodiment, the wall of the stent body and the porous layer are formed of the same material. In particular, the porous layer may be formed of a foamed material having a lower density than the density characterizing the wall of the stent body. Manufacturing of the stent may be facilitated by employing only a single material or material composition to form the stent body and the porous layer. Foaming of the porous layer material, e.g. by using air or another (gaseous) fluid, results in a lower density of the porous layer material. Compressibility and adaptability towards the inner wall of the hollow organ may be improved. As a result thereof, positioning and maintenance of the proper position after deployment of the stent may be enhanced. The wall of the stent body is preferably not based on a foamed material. The mechanical robustness representing the required resilience and a liquid-tight structure of the stent body (wall) are ensured. Thus, the wall of the stent body preferably exhibits a larger density than the foamed porous layer.

The porous layer may have an essentially homogeneous density, but may alternatively also comprise a density gradient, e.g. increasing from the inner circumference to the outer circumference of the porous layer.

To further facilitate the manufacturing of the stent, the wall of the stent body and the porous layer may preferably be manufactured as a single piece. The wall of the stent body and the porous layer may be secured to each other in a material bonding manner, e.g. by an adhesive. While the wall of the stent body and the porous layer may be formed of distinct materials or material compositions, a final curing step or final curing time period may preferably still be performed simultaneously, i.e. with the porous layer covering the outer circumference of the wall and being in contact therewith. The wall of the stent body and the porous layer may accordingly form an integral part, preferably by means of material bonding.

A single piece structure, e.g. an integral part of the wall of the stent body and the porous layer, preferably by means of material bonding, has the advantage that the structural integrity of the stent may be significantly improved and material properties may be essentially homogenous along the longitudinal extension of the stent. Furthermore, a single piece structure does essentially not require any further mechanical fixations to ensure that the porous layer remains in a secured position relative to the wall of the body. Accordingly, it may be ensured that the stent is functionally fully operable. In particular, it is ensured that portions of the stent, e.g. portions of the porous layer, do not remain within the patient's body when removing the stent.

The extension along the predefined section of the stent body is preferably configured such that the longitudinal end faces of the porous layer are covered by a liquid-tight cover. The cover may typically be represented by a sheet-like layer. The liquid-tight cover may also be connected to the respective longitudinal end of the wall or stent body. Thereby, the porous layer may be mechanically secured relative to the stent body, preferably in a form fitting manner.

In the deployed state, a longitudinal direction of the porous layer typically extends at least partially in parallel with the inner wall of the hollow organ at the target application site, i.e. a predefined anatomical region of the hollow organ exhibiting a leakage, a wound or a lesion. However, the provision of the cover at the respective end faces may enable another advantage. The porous layer may be brought into contact with the target tissue in a longitudinal direction, i.e. an inner wall of the hollow organ facing the (distal) end face of the porous layer. Thereby, an improved sealing may be ensured towards the surrounding tissue or lumen of the organ, e.g. towards a portion of the intestinal tract. In particular, leakage of body fluids may be prevented by the cover at the longitudinal end portion of the porous layer and the stent body. In other words, body fluids or chyme or stool, for example, may not enter into the porous layer or an interface or area between the porous layer and the inner wall of the hollow organ via either of the respective end portions. Such an embodiment according to the invention is advantageous for proper tissue healing.

Preferably, the cover is made of a soft and/or flexible material, e.g. a deformable plastic or rubber material. The physical characteristics of the cover may hence be chosen so as to reduce the risk of damaging the surrounding tissue. Preferably, the cover is capable of being adaptable to the structure of the surrounding tissue. Thereby, an improved sealing function as well as a level of cushioning or support for the surrounding tissue is provided.

To further improve the sealing towards the surrounding tissue, the outer circumference of the porous layer is preferably covered by the cover at longitudinal end portions of the porous layer. Accordingly, the cover extends from the respective end portion of the wall of the stent body to and over the outer circumference of the respective end portion of the porous layer via the respective longitudinal end face of the porous layer. In other words, the cover may form a type of sandwich-structure, covering the entire outer end portion of the porous layer that is not covered by the wall of the stent body. The risk of undesired leakage of e.g. body fluids or stool towards the target tissue is thereby further reduced.

An improved mechanical fixation of the porous layer to the wall of the stent body is enabled by providing an increased contacting surface. Form-fitting of the porous layer between the cover at the respective longitudinal end portions is improved. Thereby, the structural integrity of the stent may be maintained during deployment at the target tissue and removal.

As described above, the cover at the respective end faces may advantageously reduce the risk of leakage of e.g. body fluids to and from the target application site, e.g. an anastomosis. Since the cover is connected with the wall of the stent body, it may provide an additional mechanical fixation of the porous layer relative to the wall, e.g. in a form fitting manner. Mechanical fixation is thereby at least established in the longitudinal direction. It may, however, also be provided in the radial direction by enveloping the outer circumference of the porous layer at the respective end portion by the cover, as described above.

In an embodiment, the cover may extend from the respective longitudinal end of the wall as a sheet-like cover. During manufacturing of the stent (or preparation of the stent for deployment), the cover may e.g. extend (e.g. from the wall) in a tapered manner, for example radially outward, beyond the end face of the wall and may be folded back and around the porous layer at the respective end portion. The cover extends from the wall over the end face of the porous layer and over the outer circumference of the end portion of the porous layer. In other words, the sheet-like cover may be folded in a backward direction, i.e. towards to other longitudinally opposing end of the stent along the exterior of the stent. The cover thus facilitates the covering of the end face and, optionally, the outer circumference as well as the fixation of the porous layer to the wall of the stent body.

Depending on the dimensions and type of material for the cover, a loose form fitting of the porous layer may also be provided, such that e.g. radial or longitudinal forces exerting upon the porous layer may still result in a corresponding displacement. The fixation of the porous layer onto the wall by the cover at the longitudinally opposing end faces, however, ensures that, at least during deployment of the stent, the porous layer is biased between the opposing end regions. Thereby, the stent is properly implanted at the target site.

Preferably, the cover and the wall of the stent body are formed as a single piece. For example, both the cover and the wall of the stent body may be formed of a silicon-based material and the wall of the stent body and the cover may be simultaneously cured in a single mold. Thereby, mechanical fixation of the cover to the wall of the stent body may be significantly improved, requiring no separate fixation step or attachment means. This is in contrast with prior art solutions implementing a separate sealing element that is required to be secured to a portion of the stent or stent body. Such prior art solutions are not only cumbersome, but typically also result in sealing deficiencies due to insufficient attachment or loosening of the sealing element. Instead of a silicon-based material, the cover may also be formed of a polyurethane, latex, hydrocolloid, lyogel, or hydrogel. The cover preferably enables a homogenous and structurally robust sealing. It may establish a fluid-tight, i.e. air-tight and water-tight sealing.

To improve the folding of the sheet-like cover, the cover is preferably thinner than the wall of the stent body. Thereby, the cover typically exhibiting reduced resilience may adopt a more flexible character. Thereby, covering over the respective end face and potentially a predefined shape, e.g. a curvature, of the end portion of the porous layer may be advantageously enabled. The thinner sheet-like material may e.g. be formed as a foil or membrane, depending on the in situ constraints at the target site.

In another embodiment, the cover is provided in a material-bonding manner with the wall of the stent body and the porous layer. The cover may e.g. be silicon-based. The cover material may be applied at least to the end face of the porous layer and an adjacent wall portion of the stent body by dipping. The e.g. liquid cover material may hence be evenly applied on the wall and/or the end face of the porous layer by the dipping process. The cover material may engage with or may fill the pores of the porous layer after curing of the cover material. Thereby, mechanical fixation of the porous layer to the wall of the body may be significantly improved. Furthermore, manufacturing may be further facilitated, since the application of the cover material and the securing of the porous layer essentially only require a single dipping step for each respective end portion.

The longitudinally opposing end portions of the porous layer may comprise an enlarged radially outward extension. In other words, the end portions of the porous layer may comprise a larger radially outward extension than the portion of the porous layer in between said end portions, e.g. the portion (other than the end portions) exhibiting an essentially cylindrical or tubular shape. Accordingly, both opposing end portions may e.g. have a varying cross-sectional area or shape and/or exhibit a radial increase in thickness as compared to the portion of the porous layer having an essentially continuous cross-sectional area.

Preferably, the end portions of the porous layer exhibit a mushroom shape, a dome shape, a toroidal shape, or donut shape. The porous layer preferably has a barbell shape in a longitudinal section of the porous layer. A preferred shape exposes a rounded surface without sharp edges, thereby reducing the risk of tissue damage at the contact points with the hollow organ epithelium, in particular upon deployment or dislodgement of the stent body. The rounded shapes also ensure an improved fitting and sealing to the local anatomy at the application site, i.e. rendering it adaptable to the inner wall of the hollow organ. Alternatively, one or both of the longitudinally opposing end portions of the stent body or wall thereof may also exhibit any of the above described shapes, such that e.g. one of its longitudinally opposing end portions may be formed as a radially extending toroidal shape. The other longitudinally opposing end portion may e.g. comprise a similar shape, may comprise a flange-like extension in a radial direction, or may comprise no radial extension.

In this regard, an enlarged radial extension at both opposing end portions also enables the stent to be placed in such a manner that e.g. a lesion or suture of the hollow organ contacts the section of the stent between its respective end regions. Thereby, the lesion or suture is separated from the lumen of the hollow organ. Targeted application of a vacuum or drainage at the site of the lesion or suture is supported as well.

Preferably, at least one longitudinal end portion of the porous layer and the respective cover are configured for accommodating a cannula. In particular, a proximal end portion of the stent may be advantageously configured for accommodating a cannula. The cover in this regard ensures that the cannula entrance is properly sealed. Any such sealing is preferably established by applying the cover material in a semi-cured or uncured manner over at least the respective end face of the porous layer and around the cannula at its entrance site into the porous layer. Alternatively, the cover may e.g. be characterized by a predefined hole dimensioned to accommodate the cannula in a press-fitting manner and/or an additional sealant may be provided, e.g. silicon-based material comprising a hydrogel.

Accordingly, the stent preferably further comprises a cannula being accommodated by the porous layer outside of the wall. For example, the stent may comprise a cannula or other drainage means, which is accommodated alongside the stent body. It may be guided through the porous layer or along the interface established by the porous layer and the wall of the stent body, e.g. at a respective longitudinal end portion of the stent. Hence, the cannula may be preferably arranged in the space between the porous layer and the outer circumference of the wall of the stent body. The cannula may e.g. be couplable to a vacuum or negative pressure source to provide a vacuum or negative pressure in the space defined by the wall of the stent body and an inner wall of the hollow organ in the implanted and deployed state. As described above, applying a negative pressure or vacuum, i.e. pressure lower than atmospheric pressure, or a vacuum may facilitate wound healing by providing or facilitating closure of a lesion or wound being adjacent to or covered by the porous layer and/or by ensuring drainage of wound fluid or any potential contaminating liquids or semi-liquids, such as chyme or stool.

Preferably, the cannula is arranged such that a distal opening of the cannula is arranged adjacent to a distal end portion of the porous layer, e.g. at a position within a section representing 60% to 85% of the total length L of the stent. Thereby, the efficacy of the applying a negative pressure by e.g. an extracorporeal device may be improved along the longitudinal extension of the stent. In particular, the cannula's distal opening may be arranged at a position within the radially extended distal end portions of the porous layer or just proximal to that distal end portion. By one embodiment, the cannula may not only comprise its distal opening. It may additionally comprise one or more openings or holes within the wall of the cannula and located more proximally to the distal end of the cannula. Such additional openings or holes within the side wall of the cannula may also be arranged at a corresponding proximal end portion of the porous layer, e.g. at a radially extended proximal end portion of the porous layer. Depending on the required level of (negative) pressure distribution and/or drainage, one or more openings or holes within the side wall of a cannula may be foreseen between the proximal portion of the cannula at the proximal end portion of the porous layer and the distal opening of the cannula. Thus, various such additional openings or holes within the side wall of the cannula, e.g. 2 to 6, may be foreseen along the extension of the porous layer. Advantageously, the cannula is positioned as component of the stent such that the openings within the side wall of the cannula are directed radially outward.

The stent may also be equipped with more than one cannula being accommodated by the porous layer and/or within the space defined by the wall of the stent body and the porous layer. Their distal end(s) with their distal openings is advantageously positioned in such a manner that they are positioned at another site along the longitudinal extension of the stent. Thereby, the positions of the respective distal openings of each cannula is different, e.g. for applying uniform (negative) pressure conditions along the extension of the stent at the implantation site or for applying a negative pressure gradient or for applying peak negative pressure conditions at the longitudinal end portions, e.g. by cannulas positioned close to (i) the proximal and close to (ii) the distal longitudinal end portion of the stent.

Furthermore, other functions may optionally, preferably additionally, be exerted by using the cannula, e.g. enabling a rinsing or flushing at the application site, e.g. with saline or other biologically compatible fluid, or by applying a liquid or gel-like tissue sealant to facilitate healing of a lesion or leaky suture. The rinsing or flushing function may be provided by the cannula configured for applying a negative pressure. Alternatively, to enable such a rinsing or flushing function, one or more extra-cannulas may be foreseen, e.g. by fastening it/them on the stent body as describe above, in addition to the cannula(s) configured for applying a negative pressure. By such an embodiment, the stent comprises at least one cannula for applying a negative pressure and at least one extra-cannula for enabling a rinsing or flushing function.

The one or more cannula/s for applying a negative pressure and, optionally, the one or more extra-cannula/s for rinsing/flushing may be secured to the stent body by means of the cover at a respective end face of the porous layer, i.e. an insertion end face. Alternatively, or in addition, the cannula/s may also be secured to or fastened on the stent body by means of one or more sutures, by the application of an adhesive at the respective end face, and/or by an interference fit provided by the porous layer.

As described above, the application of a vacuum may allow any contents leaking into the space between the wall of the stent body and the inner wall of the hollow organ to be effectively drained by means of a negative or suction pressure. For example, a drainage of inflammatory secretions from the lesion may be carried out. Also, body fluids entering into the intermediate space may be effectively removed or sucked away from the lesion, so as to avoid improper wound healing or sepsis following leakage of e.g. chyme or stool. Such suction by applying a (small) vacuum may also result in or facilitate a sealing of the lesion or wound, e.g. at the interface of the wound and/or mucosa. Thereby, the healing process may be further supported.

In order to provide a selective reinforcement of the stent, the stent may comprise a mesh-shaped frame portion enclosing or surrounding only a portion of the stent body. The frame portion is preferably arranged so as to enclose or abut only one of the opposing longitudinal end portions of the stent body. At least the other longitudinal end portion of the stent body is hence not covered or accommodated by the frame portion. Alternatively, the mesh-shaped frame portion may extend along the entire length of the stent body. The mesh-shaped portion is preferably sandwiched by the stent body or the wall of the stent body, respectively, as the internal component and the porous layer as the external component.

According to the present invention it has been found that such configuration may be particularly advantageous for rectal applications, since sealing insufficiencies were identified to typically increase in a direction towards the rectum and sphincter muscle. In this regard, the frame portion was found to advantageously provide an increase in the sealing pressure applied towards the corresponding surrounding target tissue and/or to reduce inadvertent deformations of the stent, e.g. of the porous layer, which may particularly occur upon movement and/or (strong) contraction of the surrounding muscle tissue.

On the other hand, the portion of the stent body not being enclosed by the frame portion hence defines a free portion, which may advantageously be deployed in closer proximity to the sphincter muscle or extend towards the exterior out of the anus. The lack of such reinforcement may ensure that the corresponding portion of the stent body is less prominent and be perceived as more comfortable and less noticeable by the patient.

The frame portion is preferably arranged between the porous layer and (the wall of) the stent body. The frame portion may be secured to the porous layer and the stent body by means of material bonding, e.g. by an applied medical adhesive. Alternatively, or in addition, the frame portion may also be secured or held in place by one or more material bonding points between the porous layer and the wall of the stent body, which may accordingly extend through the mesh wire of the frame portion and provide a positive interlocking, preferably at least in the longitudinal direction.

The portion of the stent body or wall thereof being covered by the frame portion in a longitudinal direction may be between 5% and 70% of the stent body length, for example between 30 mm and 50 mm for an overall stent body length up to 250 mm to 400 mm. Preferably, the portion of the stent body being enclosed or covered by the frame portion is between 40% and 60% of the stent body length, in particular between 30 mm and 50 mm for an overall stent body length between 80 mm to 120 mm. As described above, the frame portion encloses the portion of the stent body, such that the frame portion extends along the entire circumference of the stent body. In other words, the portion of the stent body being covered is hence to be understood to correspond to a length of the stent body being covered by a continuous extension of the frame portion compared to a length of the portion of the stent body not being enclosed by the frame portion.

Preferably, the frame portion encloses one of the opposing longitudinal end portions of the stent body, such that said longitudinal end portion of the stent body is accommodated within the frame portion. The frame portion preferably comprises a shape matching the shape of the accommodated portion of the stent body. Such configuration ensures that the overall dimensioning may be reduced and an improved sealing and securing of the stent body to the frame portion may be achieved. Furthermore, in particular when the frame portion is arranged between the porous layer and the stent body, this enables that the longitudinal end face of the porous layer may be in direct contact with the cover extending from the stent body. Accordingly, the frame portion is preferably shaped such that the porous layer abuts the longitudinal end portion of the stent body being accommodated within the frame portion.

The longitudinal end portion of the stent body not being enclosed or covered by the frame portion as well as the corresponding end portion of the porous layer may be configured as described above. Alternatively, it may be provided that no liquid-tight cover is applied to the corresponding longitudinal end face of the porous layer. Such configuration may e.g. be provided, if the portion of the stent body not being enclosed by the frame portion is configured to extend towards the exterior of the patient's body, e.g. through the anus of the patient. In such configuration, the radial extension of the longitudinal end portion of the stent body may correspond to the adjacent portion of the stent body.

Alternatively, the longitudinal end portion of the stent body not being enclosed by the frame portion may radially extend as described above or preferably comprises a flange-like extension. Such shape may be particularly advantageous to provide an abutment at the sphincter muscle. The flange-like extension may hence comprise a diameter exceeding the diameter of the adjacent portion of the stent body, e.g. by 10% to 50%. In particular, for a diameter of the stent body of between 20 mm and 28 mm, the diameter of the flange-like extension may be increased by 4 mm to 12 mm. In a particularly preferred embodiment, the diameter of the stent body may be between 22 mm and 26 with the diameter of the flange-like extension being between 30 mm and 34 mm.

In order to facilitate the deployment of the stent, the frame portion is preferably formed of a resilient and self-expandable material. In particular, the frame portion may be based on or may consist of nitinol or other memory-shaping material. Due to the resilience the frame portion may hence be collapsed to facilitate insertion for deployment of the stent while the self-expanding properties facilitate deployment at the target site.

In accordance with the invention, a method for manufacturing a stent suitable for introduction into a hollow organ of the human or animal body, preferably into the gastrointestinal tract, in particular the intestine, is disclosed, comprising the steps of:
providing a wall of a stent body being preferably resilient in a radial direction and defining an inner fluid passageway from one end of the stent body to a longitudinally opposing end of the body, the wall defining a liquid-tight barrier in radial direction;
providing a resilient porous layer,
covering an outer surface of the wall along its entire circumference and along a predefined section in a longitudinal direction of said stent body with the porous layer; wherein the porous layer and the wall are adapted for applying a negative pressure toward a portion of the hollow organ; and
optionally covering at least longitudinally opposing end faces of the porous layer with a liquid-tight cover material being in contact with the respective longitudinal end of the wall.

The wall of the stent body is preferably formed of a plastics or rubber material, which is preferably silicon-based. For example, the wall of the stent body may be provided by molding and curing of the material so as to obtain e.g. a tube-like or cylindrical structure defining a liquid-tight barrier towards the inner fluid passageway. The result of providing a wall of a stent body according to the above manufacturing method is a liquid tight barrier separating the inner fluid passageway and the porous layer. The resilient porous layer may e.g. be provided as a foam structure, which facilitates adaptability to the inner wall of the target hollow organ and may improve securing the stent at the target site.

Preferably, the porous layer and the wall of the stent body are formed of the same material, wherein the porous layer is provided by foaming the material so as to obtain a layer having a lower density than the material forming the wall of the stent body. The wall of the stent body may hence exhibit a larger density, such that a predefined resilience of the stent body and its character as a liquid-tight barrier are ensured. By using the same material, manufacturing of the stent may be facilitated and the mechanical properties may be better defined.

In particular, the wall of the stent body and the porous layer may be formed as a single piece and/or the wall of the stent body and the porous layer may be connected to each other by material bonding. Preferably, the porous layer and the wall form an integral part, wherein the porous layer is formed at the outer circumference of the wall by foaming the porous layer material which essentially surrounds the wall of the stent body. In other words, the material may be provided with a predefined density for the wall of the stent body. Its density may e.g. be reduced by foaming said material, e.g. by using air, thereby forming the lower density porous layer outside of the wall. The density may hence transition from a higher density at the wall of the stent body to a lower density of the porous layer at its radially outer zone, e.g. by a (small) gradient. The porosity of the porous layer may be adjusted by the appropriate choice of the material and/or the amount of foaming, e.g. the speed, duration, and penetration depth of air, for example.

In an embodiment, the cover may be formed as a sheet-like longitudinal extension of the wall, which is folded around a respective longitudinal end portion of the porous layer to at least cover the respective end face of the porous layer. The cover may e.g. be formed as a longitudinally and/or radially outward extending portion of the wall, which preferably exhibits a reduced thickness as compared to the wall. Its reduced thickness may facilitate the application of the sheet-like cover material over the porous layer, in particular including coverage of an outer circumference of the porous layer at the respective longitudinal end portion. The sheet-like material may e.g. be applied to the porous layer in a semi-cured state. The sheet-like material is then fully cured and coats the porous layer. Alternatively, if required, the sheet-like material may also be (further) attached to the porous layer by other means, preferably by corona plasma treatment or by application of a sealant.

In a further embodiment, the cover is established by dipping the respective end face of the porous layer and the respective longitudinal end of the wall into the (liquid or semi-liquid) cover material. As described above, the dipping ensures the (semi-liquid) cover material to be evenly applied. The cover material closes the pores of the porous layer after curing of the cover material. Accordingly, mechanical fixation of the porous layer to the wall of the stent body, essentially in a single step, may be ensured or improved.

Preferably, a cannula is positioned in a space between the wall of the stent body and the porous layer and/or inserted into the porous layer. Upon inserting the cannula in the porous layer, the cannula does typically not directly contact the stent body. For example, the cannula may be inserted through a longitudinal end face, in particular a proximal end portion, of the porous layer. Thereafter, the respective end face of the porous layer and the respective longitudinal end of the wall may be dipped into the cover material. If the cannula is positioned in the space between the wall of the stent body and the porous layer, it may fastened on or secured to the wall of the stent body first. Thereafter the porous layer may be positioned on the stent body with the cannula fastened thereon. Finally, the respective end face of the porous layer and the respective longitudinal end of the wall may be dipped into the cover material.

The cannula is preferably secured to or fastened on the stent body by means of one or more sutures, by application of an adhesive, e.g. at a respective end face of the porous layer accommodating the cannula, and/or by an interference fit provided by the porous layer and/or a cover, e.g. at a respective end face of the porous layer accommodating the cannula.

The method as described above may further including the step of providing a mesh-shaped frame portion. Optionally, a mesh-shaped frame portion is arranged such that only a portion of the stent body is enclosed by the mesh-shaped frame portion. Advantageously, the mesh-shaped frame portion is arranged around the stent body prior to the step of covering the outer surface of the wall with the porous layer, such that the mesh-shaped frame portion is at least partially arranged between the porous layer and the wall of the stent body.

The features and advantages discussed with respect to the stent also apply to the method of manufacturing and vice versa.

According to another aspect, a method for sealing a leakage, a lesion or a wound of a hollow organ of a human or animal body, preferably of the gastrointestinal tract, in particular the intestine, is provided, comprising the steps of:
(a) introducing a stent according to the invention into the hollow organ, thereby enabling the stent to cover the leakage, wound or lesion site, the stent comprising at least one cannula being accommodated by the porous layer outside of the wall or in the space between the wall of the stent and the porous layer; and
(b) applying, by the cannula, a subnormal pressure at the target implantation site of the stent, thereby sucking the hollow organ onto the porous layer at the target implantation site.

The stent, being configured as a suction stent, may be introduced into the hollow organ in a compressed state and may be advantageously deployed at a target implantation site of the hollow organ using a catheter and/or endoscope. The method thus applies a stent as disclosed herein, which comprises a cannula. The sub-normal pressure applied extra-corporeally is transferred via the cannula to the stent implantation site. The inner wall of the hollow organ is sucked onto the porous layer. Thereby, body fluid passing through the hollow organ, such as chyme or stool, is prevented from contacting the lesion or wound at the implantation site of the hollow organ. As a result, wound healing is accelerated and any infection risk conveyed by the body fluid, including sepsis, is significantly reduced. By equipping the stent with e.g. another cannula, the method may comprising another step of rinsing or flushing the site of implantation in the area of the lesion or leakage site, e.g. by an isotonic solution.

### BRIEF DESCRIPTION OF THE DRAWINGS

The present invention will be readily appreciated by reference to the following detailed description when being considered in connection with the accompanying drawings in which:

FIG. 1 shows a schematic depiction of a stent according to the invention in a longitudinal section;

FIG. 2 shows a schematic depiction of a stent according to the invention in a longitudinal section according to another embodiment;

FIG. 3 schematically shows method steps for manufacturing a stent according to the invention;

FIG. 4 shows a schematic depiction of a stent according to the invention in a longitudinal section according to another embodiment with a frame portion; and

FIG. 5 shows a schematic depiction of a stent according to the invention in a longitudinal section according to another embodiment with a frame portion.

The embodiments of the Figures do not limit the invention.

### DETAILED DESCRIPTION

In the following, the invention will be explained in more detail with reference to the accompanying figures. In the Figures, like elements are denoted by identical reference numerals and repeated description thereof may be omitted in order to avoid redundancies.

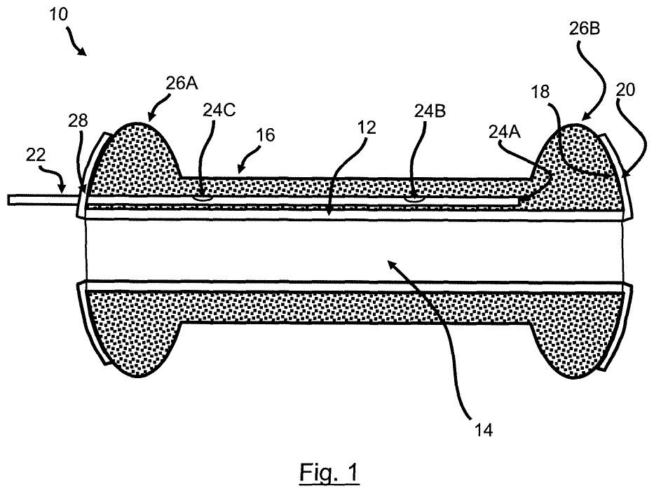

In FIG. 1, a schematic depiction of a stent 10 according to the invention is shown in a longitudinal section. The stent 10 is compressible in a radial direction and comprises a predefined resilience, such that it may be collapsed for deployment by conventional application measures. It may return to its original state at the site of deployment, exerting a (small) radial force towards the inner wall of a hollow organ at the target site. The stent 10 may optionally be (self)expandable. The stent 10 may have various dimensions, so as to be adapted to the dimensions of the hollow organ, in particular the gastrointestinal tract, preferably the esophagus, intestine, primarily rectum, sigma, colon descendens or colon transversum.

The stent 10 comprises a stent body which is essentially formed by a wall 12. According to the present, non-limiting embodiment, the stent body is formed of a plastics or rubber material and is preferably silicon-based. The wall 12 is hence formed of an advantageously resilient, compressible, and collapsible material.

The wall 12 of the stent body has an essentially tubular shape and comprises an essentially continuous cross-sectional area. It extends in a longitudinal direction, e.g. from a proximal end towards a distal end. The terms “proximal” and “distal” are to be understood so as to be closest to an insertion site and closest to an application site, respectively, delivering of the stent 10 towards the application site by an intervention.

Between longitudinal end portions (26A, B) of the wall 12, the wall 12 defines a continuous fluid passageway 14. The wall 12 is formed as a continuous and essentially homogenous liquid-tight barrier, preferably formed of a single plastics or rubber material, such that the passageway 14 is sealed-off in a radial direction by means of the wall 12. The wall 12 hence ensures that body fluids or chyme or stool may not be conveyed via or through the wall to the exterior of the stent 10, such that the risk of its contact with the exterior in the deployed state (i.e. lesion or leaky suture is significantly reduced. Potential medical complications resulting from passage of such materials may be effectively avoided.

A sealing towards the inner wall of the hollow organ is facilitated by a porous layer 16, which is indicated with the corresponding hatching. The porous layer 16 surrounds the wall 12 along its outer circumference. It may be formed of a biocompatible foam or sponge material so as to provide sufficient shapeability and conformability to the local tissue and anatomic structure at the application site. Thereby, such material may also assist in mechanically securing the stent 10 at the desired application site. As shown, the porous layer 16 comprises end portions (26A, B) having an increased radial extension, which form respective convex and/or toroidal portions. The porous layer 16 may define a barbell shape in a longitudinal section. The increased radial extensions facilitate that the stent 10 is securely held in place and may furthermore provide an improved sealing of a lesion, once the stent 10 is positioned such that the lesion is located in between the respective longitudinal end portions of the stent 10 across the e.g. gastrointestinal wall.

By means of the passageway 14 and the improved sealing provided by the end portions (26 A, B) of the porous layer 16 as well as the wall 12 of the stent body, the physiological function of the hollow organ may be re-established while lesions or leaky sutures, for example, anastomosis, may heal without any major impairment.

At the longitudinal end faces 18 of the porous layer 16, a cover 20 is foreseen, which is connected to the respective longitudinal end portion (26A, B) of the wall 12 and extends around the end face 18 so as to provide a cover in the radial direction. The cover 20 is liquid-tight, such that the cover 20 ensures the porous layer 16 to be sealed in a radial direction at the respective longitudinal end portion (26A, B). Accordingly, a sealing function may be provided with regard to an adjacent inner wall of a hollow organ facing the porous layer 16. The body fluids or chyme or stool may not enter into the intermediate space between the opposing longitudinal end portions (26A, B) on the one hand and between the wall 12 and the inner wall of the hollow organ (radial direction) on the other hand.

The cover 20 according to the present non-limiting example is formed integrally with the wall 12 and/or is connected via mechanical bonding, so as to improve the structural integrity and simplify the manufacturing. The cover 20 is formed as a sheet-like material, which extends from the wall 12 and is shown as being connected in a material bonding manner, as indicated with the respective small lines at the interface between the wall 12 and the cover 20. The sheet-like material has a lower thickness than the wall 12, which facilitates application of the cover 20 to the porous structure of the porous layer 16 at the respective end face 18. The sheet-like cover 20 may e.g. be applied in a semi-cured state to the respective end face 18. Alternatively, or in addition, the sheet-like cover 20 may be attached to the respective end face 18 e.g. by means of corona plasma treatment, which has been found to provide a particularly effective bonding, e.g. for silicon-based materials.

Also shown in FIG. 1 is a cannula 22, which is received at an (a proximal) end region and is introduced via the cover 20 and is accommodated along the portion of the wall 12 between the proximal end region and the opposing distal end region. The cover 20 comprises an entry opening (28) dimensioned and adapted to receive the cannula 22, such that the cannula 22 is fully surrounded by the cover material 20, preferably in a liquid-tight manner. By means of the cannula 22, a negative pressure or suction pressure may be applied between the radially outward extending longitudinal end portions (26A, B) of the porous layer 16, such that an inner wall of the hollow exhibiting a lesion may be properly closed. As shown, the cannula 22 is preferably accommodated in the porous layer 16 such that a distal opening (24A) of the cannula 22 is arranged at the level of or close to a distal longitudinal end portion (26B) of the porous layer 16, advantageously at a section defined as 60% to 85% of the entire stent length L, with the proximal end of the stent being 0%. However, the cannula may, instead also be fastened on the wall of the stent body (without any porous layer material being located between the cannula and the wall of the stent body (not shown)). In the present, non-limiting example, the distal opening (24A) of the cannula 22 is accordingly arranged in proximity of the distal enlarged radially outward extension of the porous layer 16. While the porous layer 16 may facilitate proper positioning and securing of the stent 10 relative to the inner wall of the hollow organ, it may also ensure that the inner wall of the hollow organ is not brought into direct contact with the cannula 22. Thereby, the suction force being applied to the inner wall of the hollow organ may be controlled and more evenly distributed: Inadvertent suction of the inner wall may be effectively avoided.

At least one additional opening, e.g. 2 to 6 additional openings (24B and C) may be provided as holes of the wall of the cannula (22). The may be foreseen at sites of the sidewall which are in contact with the porous layer (16). They may ensure that the sub-normal pressure is more evenly applied along the length of the stent, thereby ensuring that the wall of the hollow organ is sucked onto the stent at the implantation site.

The embodiment in FIG. 1 shows one single cannula (22). However, more than one cannula (22) may be foreseen. In addition to cannula (22), the stent according to the embodiment of FIG. 1 may comprise at least one extra-cannula for rinsing and flushing (30) (not shown in FIG. 1).

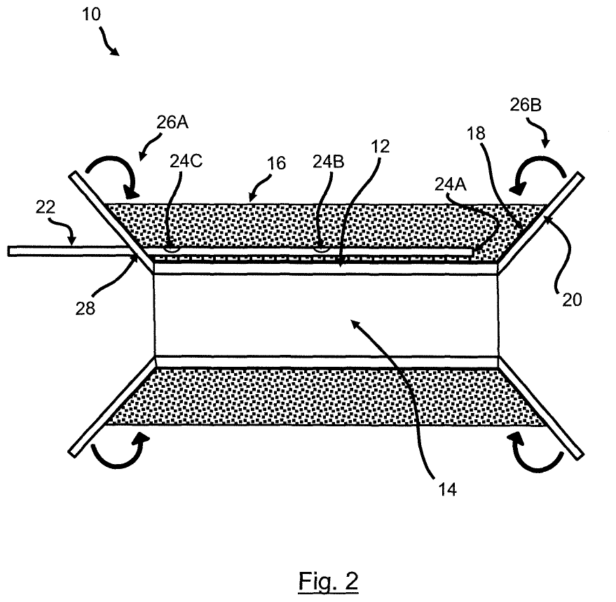

In FIG. 2, a stent 10 according to another embodiment is schematically shown. According to the exemplary embodiment, the porous layer 16 (indicated with the hatching) does not comprise an increased radially outward extension at the opposing longitudinal end portions (26A, B). Furthermore, the porous layer 16 may optionally extend beyond the wall 12 in the longitudinal direction, which may be advantageous e.g. to support an extension of a cannula (not shown) without requiring an enlarged dimensioning of the wall 12. This may be particularly advantageous, if the wall 12 of the stent body comprises a larger resilience, such that stent 10 as a whole may be better adapted to the anatomical landscape of the target site.

Furthermore, a cover 20 extending from the wall 12 is present at the respective end faces 18. As indicated by the thick semi-circular arrows, the cover 20 may be folded over the porous layer 16 so as to cover an outer circumference of the porous layer 16 at a respective longitudinal end portion (26A, B). Thereby, a sealing function of the cover 20 may be further improved, ensuring no materials to be conveyed through the lumen of the stent, preferably no fluids, such as chime or stool, may pass the longitudinal end face 18 and the adjacent inner wall of the hollow organ. The folding may be performed during manufacturing, e.g. in a semi-cured state, but may also be performed prior to collapsing the stent 10 for subsequent deployment. Prior to deployment, the cover 20 may optionally be fixed to the outer circumference of the porous layer 16, e.g. using corona plasma treatment, application of a sealant or adhesive, or by other suitable means. The level of fixation of the cover 20 to the outer circumference of the porous layer may depend on the particular requirements of the stent 10 for the respective therapeutic application and/or on the configuration of the stent 10 as a whole. For example, further means for attachment of the cover 20 to the outer circumference of the porous layer 16 may be provided during the deployment of the stent 10 and/or the treatment of the target site, e.g. a lesion.

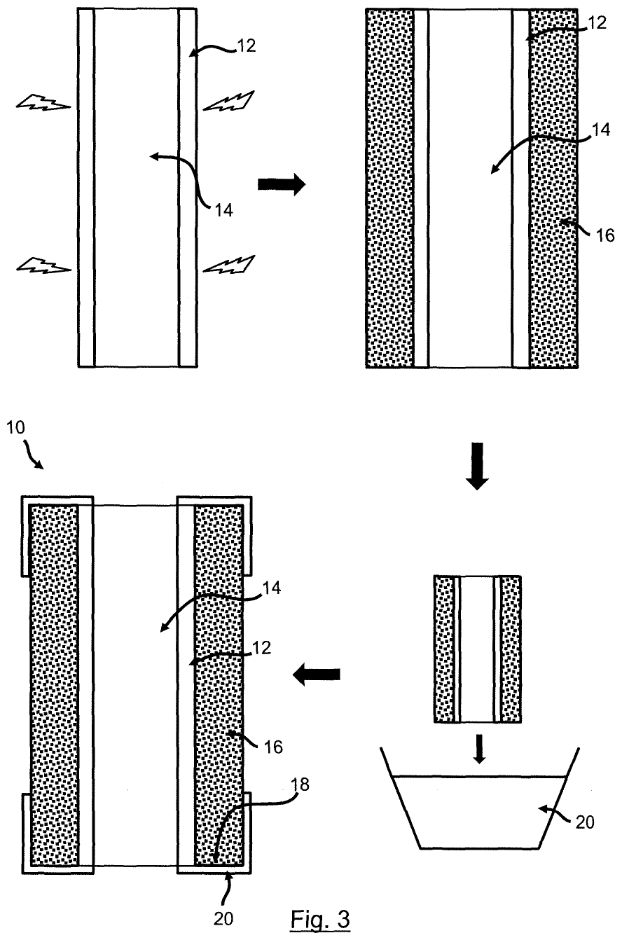

In FIG. 3, a sequence of method steps for manufacturing a stent 10 according to the invention are schematically depicted. Accordingly, in a first step indicated in the top left, a stent body is provided comprising a wall 12, e.g. of a cylindrical or tube-like shape. The wall may have been formed e.g. by molding and is preferably formed of a silicon-based material. Alternatively, the wall 12 may also have been formed by extrusion. However, molding may be particularly advantageous for softer plastic or rubber materials. It may reduce the amount of material required to form the wall 12. As shown, the wall 12 defines an inner fluid passageway 14 between longitudinally opposing end portions (26A, B), which is sealed in a fluid-tight manner in a radial direction by means of the wall 12, as described above.

While a porous layer 16 may be provided separately and may be applied along the outer circumference of the wall 12, according to the present non-limiting example, the porous layer 16 is formed by foaming of the excess material of the wall at its outer circumference, as indicated by the corresponding symbols in the top left panel. Accordingly, a porous layer 16 is formed on top of the wall 12, i.e. surrounding the wall 12 and extending in a radially outward direction, as indicated in the top right panel with the corresponding hatching.

After the porous layer 16 has been formed, which may e.g. exhibit a density gradient and/or an increased radially outward extension at the opposing longitudinal end portions (26A, B) (as shown in FIG. 1), the respective longitudinal end portions (26A, B) of the wall 12 and porous layer 16 are dipped into a liquid material so as to form the cover 20, as indicated in the bottom right panel. Thereby, the cover 20 is evenly applied along the respective end faces 18 and is simultaneously brought into contact with both the wall 12 and, according to the example, the outer circumference of the porous layer 16 at the respective end portion (26A, B).

The result of the application of the material to form the cover material 20 via dipping and after corresponding curing is depicted in the bottom left panel. The wall 12, the cover 20, and the porous layer 16 may hence be formed as a single piece and/or are secured to each other by means of material bonding. An improved mechanical stability results therefrom. It may significantly facilitate manufacturing, since essentially no further mechanical attachments are required. Furthermore, as shown, an inner wall of a hollow organ positioned between the opposing longitudinal end portions (26A, B) may be effectively sealed towards the fluid passageway 14 by means of the cover 20 and the wall 12, such that contaminations may be avoided to the largest extent.

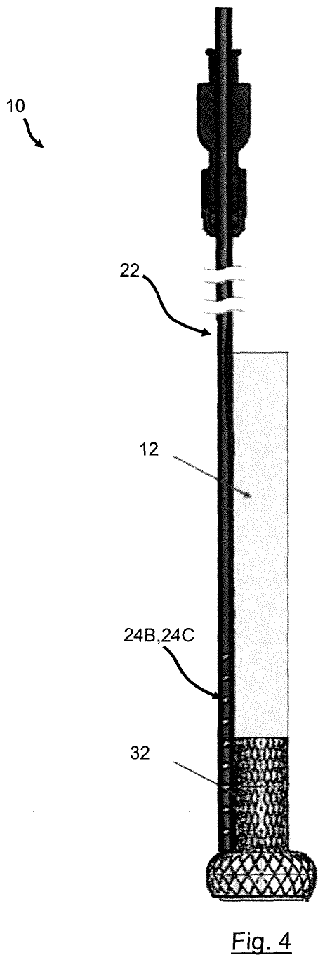

FIG. 4 shows a schematic depiction of a stent 10 according to the invention in a longitudinal section according to another embodiment with a frame portion 32. The stent 10 is depicted without the porous layer 16, which in the assembled state defines the exterior of the stent 10. As shown, the mesh-like frame portion extends circumferentially around one longitudinal end portion of the stent body, which in the present, non-limiting example is defined having a mushroom or toroidal shape. Accordingly, the longitudinal end portion of the stent body is enclosed by and accommodated within the frame portion 32.

As shown, the frame portion 32 comprises a shape matching the shape of the portion of the stent body accommodated therein, which enables that the frame portion 32 may be in direct contact with or even be integrated within the stent body. The overall mushroom or toroidal shape of the longitudinal end portion enables that a longitudinal end face of the porous layer may abut the longitudinal end portion, thereby delimiting any inadvertent movement of the porous layer in the longitudinal direction while furthermore providing an improved sealing at the corresponding longitudinal end face of the porous layer.

In the present example, the portion of the stent body being enclosed by the frame portion 32 corresponds to about 10% to 15% of the overall length of the stent body. However, it will be understood that such ratio may vary depending on the respective application of the stent 10. The extended portion of the stent body not being enclosed by the frame portion 32 may e.g. be advantageous for applications in proximity to the rectum or sphincter muscle, wherein the portion extending towards the exterior of the patient may comprise an overall reduced rigidity compared with the portion being enclosed by the frame portion 32. For such exterior portion, a cover of the longitudinal end face of the porous layer may be considered optional.

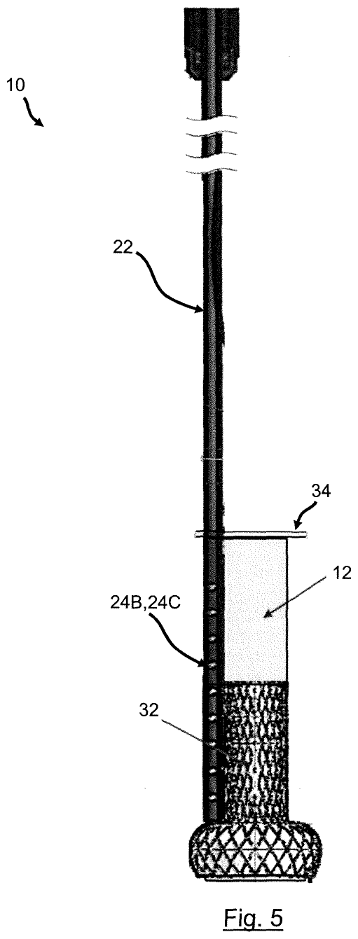

An alternative embodiment of a stent 10 according to the invention with a frame portion 32 is depicted in FIG. 5, wherein the porous layer is also not shown for illustration purposes. The porous layer may, however, likewise be arranged around the frame portion 32 and the wall 12 of the stent body so as to essentially form the exterior of the stent 10. In this embodiment, the portion of the stent body being enclosed by the frame portion 32 corresponds to about 40% to 60% of the overall length of the stent body. Furthermore, the overall length of the stent body and stent 10 is reduced compared with the embodiment depicted in FIG. 4. At the longitudinal end portion of the stent body not being enclosed by the frame portion 32, the stent body or wall 12 thereof defines a flange-like extension 34. Such flange-like extension 34 may also be particularly advantageous for applications at the rectum, wherein exterior extensions of the stent body may need to be avoided or be limited. This also reduces potential unpleasant perception by the patient. The flange-like extension 34 may be particularly dimensioned to abut the sphincter muscle or anus, thereby supporting the securing and proper placement of the stent 10. Furthermore, the flange-like extension 34 may form the cover for the longitudinal end face of the porous layer, such that the embodiment may provide an improved sealing within the hollow organ.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

### LIST OF REFERENCE NUMERALS

10 Stent
12 Wall
14 Passageway
16 Porous layer
18 End face
20 Cover
22 Cannula
24A Distal opening of cannula (22)
24B,C Openings at the wall of the cannula (22)
26A Proximal longitudinal end portion
26B Distal longitudinal end portion
28 Entry opening of the cover for cannula
30 Extra-cannula for rinsing and flushing
32 Frame portion
34 Flange-like extension

1. A stent (10) for introduction into a hollow organ of the human or animal body, preferably into the gastrointestinal tract, in particular the intestine, comprising
a stent body being preferably resilient in a radial direction and having a wall (12) defining an inner fluid passageway (14) from one end of the stent body to a longitudinally opposing end of the stent body, and
a resilient porous layer (16), the porous layer (16) covering an outer surface of said wall (12) along its entire circumference and along a predefined section in a longitudinal direction of said stent body,
wherein the porous layer (16) and the wall (12) are adapted for applying a negative pressure at the hollow organ implantation site, wherein the wall (12) forms a liquid-tight barrier separating the inner fluid passageway (14) and the porous layer (16) and wherein at least longitudinally opposing end faces (18) of the porous layer (16) are covered by a liquid-tight cover (20) being connected to the respective longitudinal end of the wall (12).

2. The stent (10) according to claim 1, wherein at least one longitudinal end portion (26A, B) of the porous layer (16) and the at least one cover (20) thereof are configured for accommodating at least one cannula (22).

3. The stent (10) according to any of the preceding claims, further comprising at least one cannula (22) being accommodated by the porous layer (16) outside of the wall (12).

4. The stent (10) according to claim 3, wherein a distal opening (24) of the cannula (22) is arranged within a section of the porous layer (16) between opposing longitudinal end portions (26A, B) of the porous layer (16), preferably adjacent to a distal longitudinal end portion (26B) of the porous layer (16).

5. The stent (10) according to any of claims 2 to 4, wherein at least one cannula (22) is adapted for applying a negative pressure.

6. The stent (10) according to any of claims 2 to 5, wherein at least one extra-cannula (30) is configured for rinsing and/or flushing.

7. The stent (10) according to any of the preceding claims, wherein the outer circumference of the porous layer (16) is covered with the cover (20) at longitudinal end portions (26 A, B) of the porous layer (16).

8. The stent (10) according to any of the preceding claims, wherein the wall (12) of the stent body and/or the porous layer (16) are formed of a plastic material or rubber material.

9. The stent (10) according to any of the preceding claims, wherein the wall (12) of the stent body and/or the porous layer (16) are formed of a silicon-based material, preferably comprising or consisting of silicone.

10. The stent (10) according to any of the preceding claims, wherein the wall (12) of the stent body and the porous layer (16) are formed of the same material.

11. The stent (10) according to claim 10, wherein the porous layer (16) is formed of a foamed material having a lower density than the wall (12) of the stent body.

12. The stent (10) according to claim 10 or 11, wherein the wall (12) of the stent body and the porous layer (16) are formed as a single piece.

13. The stent (10) according to any of the preceding claims, wherein the wall (12) of the stent body and the porous layer (16) are secured to each other in a material bonding manner.

14. The stent (10) according to any of the preceding claims, wherein the cover (20) extends from the respective longitudinal end of the wall (12) as a sheet-like material.

15. The stent (10) according to any of the preceding claims, wherein the cover (20) and the wall (12) of the stent body are formed as a single piece.

16. The stent (10) according to claim 14 or 15, wherein the cover (20) is thinner than the wall (12) of the stent body.

17. The stent (10) according to any of claims 1 to 13, wherein the cover (20) is provided in a material-bonding manner with the wall (12) of the stent body and the porous layer (16).

18. The stent (10) according to any of the preceding claims, wherein longitudinally opposing end portions (26A, B) of the porous layer (16) comprise an enlarged radially outward extension.

19. The stent (10) according to claim 18, wherein, in a longitudinal section of the porous layer (16) each respective end portion (26A, B) has a mushroom shape, a dome shape, a toroidal shape, or donut shape, and/or the porous layer (16) has a barbell shape.

20. The stent (10) according to any of the preceding claims, comprising a mesh-shaped frame portion (32) enclosing only a portion of the stent body.

21. The stent (10) according to claim 20, wherein the frame portion (32) encloses or abuts only one of the opposing longitudinal end portions of the stent body.

22. The stent (10) according to claim 20 or 21, wherein the portion of the stent body being enclosed by the frame portion (32) is between 40% and 60% of the stent body length.

23. A method for manufacturing a stent (10) suitable for introduction into a hollow organ of the human or animal body, preferably into the gastrointestinal tract, in particular the intestine, comprising the steps of:
providing a wall (12) of a stent body being preferably resilient in a radial direction and defining an inner fluid passageway (14) from one end of the stent body to a longitudinally opposing end of the stent body, the wall (12) defining a liquid-tight barrier in radial direction;
providing a resilient porous layer (16) and
covering an outer surface of the wall (12) along its entire circumference and along a predefined section in a longitudinal direction of said stent body with the porous layer (16);
wherein the porous layer (16) and the wall (12) are adapted for applying a negative pressure toward a portion of the hollow organ; and
optionally, covering at least longitudinally opposing end faces (18) of the porous layer (16) by a liquid-tight cover (20) being connected to the respective longitudinal end of the wall (12).

24. The method according to claim 23, wherein the porous layer (16) and the wall (12) of the stent body are formed of the same material and wherein the porous layer (16) is provided by foaming said material so as to obtain a layer having a lower density than the wall (12) of the stent body.

25. The method according to claim 23 or 24, wherein the wall (12) of the stent body and the porous layer (16) are formed as a single piece and/or wherein the wall (12) of the stent body and the porous layer (16) are secured to each other by material bonding.

26. The method according to any of claims 23 to 25, wherein the cover (20) is formed as a sheet-like longitudinal extension of the wall (12) and is folded around a respective longitudinal end portion (26A, B) of the porous layer (16) to at least cover the respective end face (18) of the porous layer (16).

27. The method according to any of claims 23 to 25, wherein the cover (20) is applied by dipping the respective end face (18) of the porous layer (16) and the respective longitudinal end of the wall (12) into the cover material (20).

28. The method according to any of claims 23 to 27, wherein a cannula (22) is positioned in the space between the wall (12) and the porous layer (16) and/or inserted in the porous layer (16).

29. The method according to claim 28, wherein the cannula (22) is secured to the stent body by means of one or more sutures, by application of an adhesive at a respective end face (18) of the porous layer (16) accommodating the cannula (22), and/or by an interference fit provided by the porous layer (16) and/or a cover (20) at a respective end face (18) of the porous layer (16) accommodating the cannula (22).

30. The method according to any of claims 23 to 29, further including the step of providing a mesh-shaped frame portion (32) and, optionally, arranging said mesh-shaped frame portion (32) such that only a portion of the stent body is enclosed by the mesh-shaped frame portion (32).

31. The method according to claim 30, wherein the mesh-shaped frame portion (32) is arranged around the stent body prior to the step of covering the outer surface of the wall (12) with the porous layer (16), such that the mesh-shaped frame portion (32) is at least partially arranged between the porous layer (16) and the wall (12) of the stent body.

32. A method for sealing a leakage or a lesion of a hollow organ of a human or animal body, preferably of the gastrointestinal tract, in particular the intestine, comprising the steps of:
(a) introducing a stent according to any of claims 1 to 22 into the hollow organ, thereby enabling the stent to cover the leakage or lesion site, the stent comprising at least one cannula being accommodated by the porous layer outside of the wall or in the space between the wall of the stent and the porous layer; and
(b) applying, by the cannula, a subnormal pressure at the target implantation site of the stent, thereby sucking the hollow organ onto the porous layer at the target implantation site.

33. The method according to claim 32, wherein the stent is introduced into the hollow organ in a compressed state and is deployed at the target implantation site of the hollow organ using a catheter and/or endoscope.
